(19) United States
(12) Patent Application Publication
(10) Pub. No.: US 2009/0136582 A1
(43) Pub. Date: May 28, 2009

(54) COLLOIDAL MAGNETIC NANOBIOPARTICLES FOR CYTOTOXICITY AND DRUG DELIVERY

(76) Inventors: Ralph M. Albrecht, Belleville, WI (US); Irawati Kandela, Madison, WI (US)

Correspondence Address: WARF/BHGL, P.O. Box 10395, Chicago, IL, 60610, US

(21) Appl. No.: 12/184,492

(22) Filed: Aug. 1, 2008

Related U.S. Application Data
(60) Provisional application No. 60/963,250, filed on Aug. 3, 2007.

(51) Int. Cl. A61K 9/51; A61P 43/00

(52) U.S. Cl. 424/490; 977/906

(57) ABSTRACT

The present invention provides systems that include magnetic colloidal particles of differing elemental compositions with differing morphologies. The colloidal particles comprise a magnetic material and a shell that surrounds the magnetic material. The colloidal particles can be inductively heated using a magnetic field. The present invention also provides applications of such colloidal particles, such as methods for cytotoxicity and for drug delivery.

### CROSS-REFERENCE TO RELATED APPLICATIONS

This invention claims priority to U.S. Provisional Patent Application Ser. No. 60/963,250, filed Aug. 3, 2007, which is herein incorporated by reference.

### GOVERNMENT INTERESTS

This invention was made with United States government support awarded by the National Institutes of Health, grants Nos. GM63001 and GM67244. The United States government has certain rights in this invention.

### FIELD OF THE INVENTION

The present invention relates generally to the field of colloidal magnetic nanobioparticles and methods of use thereof. More particularly, the present invention provides novel compositions and methods for cell toxicity (cytotoxicity) and for drug delivery.

### BACKGROUND

A variety of currently employed therapeutic modalities attempts to eliminate or minimize harmful cell types. Treatments for cancer (malignant cells), autoimmune diseases such as lupus and rheumatoid arthritis (auto-reactive cells), transplant rejection (allo-reactive cells), infectious diseases such as AIDS, malaria, or candidiasis (viral infected cells, plasmodium parasites, yeast)—all rely on methodologies to kill or inactivate the cell type that causes the disease. Most current therapies involve use of chemical agents such as antibiotics and chemotherapeutic compounds. Problems with this approach include cytotoxicity to the host and development of resistance to the chemical agent by the target cells or parasitic organisms. In the former, levels of agent sufficient to eliminate the target cells may exceed levels that can be tolerated by the host; in the latter, the disease can return, resistant to the therapy.

Certain therapeutic approaches include the use of ligand or antibody to target specific cytotoxic or inhibitory compounds toward specific target cells. The use of ligand or antibody increases the relative levels of agent at the specific target cells. However, nonspecific accumulation of the antibody-drug-carrier complex in organs such as the liver, spleen, or kidney can result in unacceptably high levels of the cytotoxic agent in these tissues.

Colloidal particles have found myriad uses from biology to electronics. Colloidal gold, for example, has found particular use in biological labeling for localization of cell components at the microscopic and the ultrastructural level. In such use, colloidal gold is conjugated to a wide variety of biologically active molecules, i.e., biomolecules, such as antibodies, antibody fragments, ligands, ligand fragments, or other molecular species that bind specifically to cellular targets, and can be used in vitro and in vivo for tracking and labeling studies (Albrecht et al., 1992, Methods in Enzymology 215: 456-479; Albrecht et al., 1993, In: Immunocytochemistry: A Practical Approach, Beesley, ed., pp 151-176, Oxford University Press, Oxford).

Colloidal gold particles can conveniently be made in sizes ranging from about 1 nm to about 150 nm in diameter. Depending on the particle size, colloidal gold is visible, or can be enhanced to be visible, in light microscopy, electron microscopy, x-ray microscopy or scanning force microscopy. Larger particles are most useful for applications where ease of detection is important. Medium sized particles (10-20 nm) are still large enough for single particles to be detected by certain types of light microscopy (LM) and are resolvable by scanning electron microscopy (SEM) and transmission electron microscopy (TEM). Particles as small as about 1 nm are detectable by TEM.

Colloidal particles have been used for correlative methodology studies employing fluorescence-based light microscopy, interference-based light microscopy, scanning electron microscopy, transmission electron microscopy, atomic force microscopy, instrumental neutron activation analysis, and inductively coupled plasma mass spectrometry. Methodologies have been developed for the synthesis of nanoparticles of different elemental compositions and different shapes for use in a variety of applications. This includes colloidal particles made of gold, palladium, platinum, rhodium, ruthenium, silver, and iron (Meyer and Albrecht, 2000, Microscopy and Microanalysis 6, Suppl. 2; Meyer and Albrecht, 2001, Microscopy and Microanalysis 7, Suppl. 2; Meyer and Albrecht, 2002, Microscopy and Microanalysis 8, Suppl. 2; Meyer and Albrecht, 2003, Microscopy and Microanalysis 9, Suppl. 2; and U.S. Pat. No. 6,699,507). Absorption and whole body distribution of variously sized gold nanoparticles from 4 nm through 60 nm in mice has also been studied (Hillyer and Albrecht, 2001, J. Pharmaceut. Sci. 12: 1927-1936).

The advances in cancer and nanotechnology have contributed toward the fabrication of a new generation of less invasive or non-invasive therapeutic tools. For example, magnetic nanoparticles and a method for in vitro lysis of cancer cells using DC magnetic field are disclosed in U.S. Pat. No. 6,514,481 and in Bergey et al., 2002, Biomedical Microdevices 4: 293-299).

### BRIEF SUMMARY

Systems for killing cells are provided, which include: a) colloidal particles with sizes that range from about 1 nm to about 100 nm, which comprise magnetic material, shells that surround the magnetic material, and biomolecules attached to the shells; and b) a magnetic field generator for inductively heating the colloidal particles. When the colloidal particles associate with the cells and are inductively heated using the magnetic field generator, the heated colloidal particles kill the cells. These systems may not kill other cells that are in contact with the cells that are associated with the colloidal particles. The systems may include colloidal particles with shapes that are spherical, cuboidal, pyramidal, lobate, or geodesic. The systems may include shells of conjugates that include thermosensitive polymers. The biomolecules may be cell-specific. The systems may include binding of the biomolecules to other biomolecules that are both membrane molecules and specific for the cells. The systems may include cores that comprise magnetic material selected from the group consisting of iron, cobalt, zinc, or nickel. The systems may include shells that comprise metals selected from the group consisting of gold, silver, palladium, platinum, rhodium, molybdenum and ruthenium. The systems may include biomolecules that are proteins, peptides, antibodies, nucleic acids, antigens, glycoconjugates, cellular surface molecules, ligands, cellular components, toxins, or drugs.

Methods for killing cells are provided, which include: a) providing colloidal particles with sizes that range from about 1 nm to about 100 nm, which include a core that comprises a magnetic material, a shell that surrounds the core, and biomolecules attached to the shell; b) contacting the colloidal particles with the cells such that the colloidal particles associate with the cells; and c) heating the colloidal particles using magnetic fields so that the heated colloidal particles kill the cells. In the practice of the methods, the colloidal particles may have spherical, cuboidal, pyramidal, lobate, or geodesic shapes. In the practice of the methods, the biomolecules may bind to other biomolecules that are both membrane molecules and specific for the cells. In the practice of the methods, other cells, which are in contact with the cells that are associated with the colloidal particles, are not killed. In the practice of the methods, the colloidal particles may include cores that comprise magnetic material selected from the group consisting of iron, cobalt, zinc, or nickel; the colloidal particles may include shells that comprise metals selected from the group consisting of gold, silver, palladium, platinum, rhodium, molybdenum and ruthenium; the biomolecules may include proteins, peptides, antibodies, nucleic acids, antigens, glycoconjugates, cellular surface molecules, ligands, cellular components, toxins, or drugs.

Delivery systems for delivering biomolecules to cells are provided. The delivery systems include: a) colloidal particle-biomolecule conjugates, where the colloidal particles have sizes that range from about 1 nm to about 100 nm, and the colloidal particles comprise a core that includes magnetic material, and a shell that surrounds the core, where the biomolecules may be proteins, peptides, antibodies, nucleic acids, antigens, glycoconjugates, cellular surface molecules, ligands, cellular components, toxins, or drugs; and b) administration means for administering the colloidal particle-biomolecule conjugates to the cells. The administration means may include heating the magnetic material using a magnetic field. These systems may not deliver biomolecules to other cells that are in contact with the cells that are associated with the colloidal particle-biomolecule conjugates, when these are administered to the cells. The systems may include binding of the biomolecules to other biomolecules that are both membrane molecules and specific for the cells. The systems may include cores that comprise magnetic material selected from the group consisting of iron, cobalt, zinc, or nickel. The systems may include shells that comprise metals selected from the group consisting of gold, silver, palladium, platinum, rhodium, molybdenum and ruthenium. The delivery systems may include colloidal particles where the colloidal particles have shapes that are spherical, cuboidal, pyramidal, lobate, or geodesic. The delivery system may include shells with thermosensitive polymers.

Methods for delivery of biomolecules to specific cells are provided. The methods include: a) providing colloidal particles with sizes that range from about 1 nm to about 100 nm, which include a core that comprises a magnetic material, a shell that surrounds the core, and biomolecules attached to the shell; b) contacting the colloidal particles with the cells such that the colloidal particles associate with the cells; and c) heating the colloidal particles using a magnetic field so that the heated colloidal particles deliver the biomolecules to the cells. In the practice of the methods, the biomolecules may bind to other biomolecules that are both membrane molecules and specific for the cells. In the practice of the methods, the biomolecules are not delivered to other cells that are in contact with the cells that are associated with the colloidal particles. In the practice of the methods, the colloidal particles may include cores that comprise magnetic material selected from the group consisting of iron, cobalt, zinc, or nickel; the colloidal particles may include shells that comprise metals selected from the group consisting of gold, silver, palladium, platinum, rhodium, molybdenum and ruthenium; the biomolecules may include proteins, peptides, antibodies, nucleic acids, antigens, glycoconjugates, cellular surface molecules, ligands, cellular components, toxins, or drugs.

### BRIEF DESCRIPTION OF THE DRAWINGS

The invention can be better understood with reference to the following drawings and description. The components in the figures are not necessarily to scale, emphasis instead being placed upon illustrating the principles of the invention.

FIG. 1 is a schematic diagram of a magnetite/gold (core/shell) nanobioparticle with a conjugated biomolecule (A) and with three conjugated antibodies (B).

FIG. 2 is a graph illustrating characterization of gold-coated iron particles using UV-Vis spectroscopy. “cAu₁₈” indicates 18 nm colloidal gold particles; “cFe₁₅” indicates 15 nm colloidal iron particles; “Core/shell” indicates, respectively, the core and the shell of the nanobioparticles.

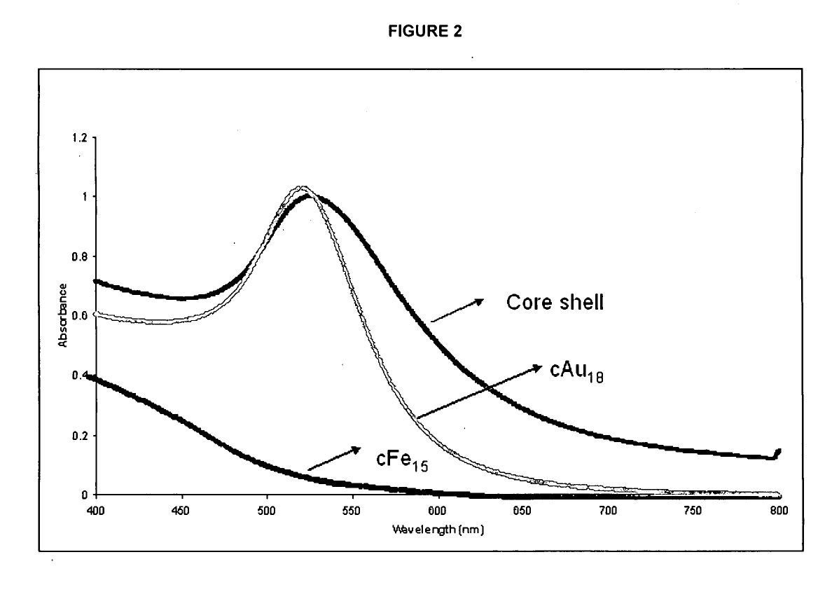

FIG. 3 is a graph illustrating characterization of gold-coated iron particles using parallel electron energy loss spectroscopy (PEELS).

FIG. 4 is a graph illustrating characterization of gold-coated iron particles using X-ray photoelectron spectroscopy (XPS).

FIG. 5 is an electron micrograph of a cell taken after seven minutes of magnetic field exposure. The arrows indicate some of the multiple core/shell particles (arrows) at the periphery of the expanding membrane holes.

FIG. 6 is a graph showing the percent tumor cell death vs. time of exposure to the oscillating magnetic field.

FIG. 7 (A, B) shows SEM images of Fe/Au (core/shell) labeled cells.

FIG. 8 shows lower (A, B) and (C, D) magnification SEM images of mixed co-cultured normal prostate cells (TCL) and prostate cancer cells (T) treated according to the methods of the present invention.

FIG. 9 (A-F) shows SEM images of SV-40 infected cells treated according to the methods of the present invention.

FIG. 10 (A-D) shows SEM images of yeast cells.

FIG. 11 shows an SEM image of a prostate cancer cell taken with S-900 at 200,000× magnification, and treated according to the methods of the present invention.

### DETAILED DESCRIPTION OF THE PRESENTLY PREFERRED EMBODIMENTS

The present invention relates to colloidal particles of differing elemental compositions with differing morphologies and to methods of preparing same. The invention is particularly well-suited for use of the colloidal particles as drug carriers, for targeting specific cell types, and for killing specific cells (or specific cell types).

As used herein, the term “nanobioparticle” refers to a particle having one or more dimensions of the order of between about 1 nm and 100 nm, to which one or more biomolecules have been attached.

As used herein, the term “cM” refers to “colloidal metal”; for example, cAu refers to colloidal gold. The number following “cM” indicates the size of the nanobioparticles in nm. For example, “cAu₁₈” indicates 18 nm colloidal gold particles (e.g., if spherical, colloidal gold particles with 18 nm diameter); “cFe₁₅” indicates 15 nm colloidal iron particles.

The terms “colloid” or “colloidal dispersion” refer to a heterogeneous mixture that visually appears to be a homogeneous solution. In a colloid, the dispersed phase is made of tiny particles or droplets that are distributed evenly throughout one continuous phase. The size of the dispersed phase particles or droplets is typically between one nanometer (1 nm) and one hundred nanometers (100 nm). The colloidal particles may have various shapes, including shapes that are spherical, oval, lobate (e.g. asterisk-like or star-like), cuboidal, pyramidal, or geodesic (e.g. multifaceted such as soccer ball-shaped).

The present invention contemplates the use of “magnetic” materials. Various types of inductively heatable magnetic materials can be used for practicing the invention. These include, for example, metals and metal oxides, such as elemental iron, iron ore, various iron oxides, the minerals hematite, magnetite, taconite, and lodestone, zinc, nickel, the rare earth metals gadolinium and dysprosium, as well as graphite, cobalt, or other magnetic materials, as disclosed in U.S. Pat. No. 6,774,346, which is incorporated herein by reference. Other (ferro)magnetic materials useful for practicing the present invention include man-made products based on naturally magnetic elements, e.g. some steels. Various magnetic alloys, such as magnetic iron-nickel alloys, can particularly be used for practicing the present invention. As well, a variety of useful superparamagnetic materials, disclosed in U.S. Pat. No. 5,411,730, which is incorporated herein by reference, can be used for practicing the present invention. Magnetite, in particular, is non-toxic and can readily be used. In general, any magnetic material can be used as long as it can be heated through the application of an electromagnetic field. Combinations of two or more magnetic materials may be used as well.

In one embodiment, the present invention provides methods for making colloidal particles of differing elemental composition. The methods include making colloidal particles of a metal including gold, silver, and platinum, by reducing a solution of a compound of the metal with a reducing agent. The reducing agent includes tannic acid, sodium citrate, ferrous sulfate, ascorbic acid, sodium borohydride, hydrogen, ethanol, methanol or combinations thereof. The incubation period/reaction time for formation of the colloidal particles ranges from virtually instantaneous to about 6 hours. The incubation/reaction temperature ranges from about 0° C. to about 100° C. The metal compound is typically a salt, suitably—but not limited to—a nitrate or a chloride.

The term “biomolecule” refers to any biological substance or material such a peptide, protein, antibody, antigen, nucleic acid, conjugate or biostructure or a fragment thereof, and also includes cellular components, glycoconjugates, cellular surface molecules, a fragment of any of these, or a therapeutic substance, for example, a drug or other chemical substance intended for use in diagnosis, cure, treatment or prevention of a pathological condition, or any other ligand used in ligand-host interactions. For example, antibodies suitably adsorbed to colloidal particles of the present invention include but are not limited to IgG or Fab fragment thereof; non-antibody proteins include but are not limited to fibrinogen, albumin and insulin. Non-protein molecules suitably include, e.g., lipopolysaccharides and lectins. The biomolecules are attached to the particles of the present invention. The biomolecules may be attached to the particles in a variety of ways, including adsorption and conjugation, either covalently or non-covalently.

The term “conjugation” refers to the binding of a metallic colloidal particle to another species, particularly a biomolecule, such as a protein, an antibody, a toxin, or a drug molecule, through noncovalent forces, which include but are not limited to adsorption, hydrophobic interactions, and van der Waals forces. Conjugation of biomolecules to the particles of the present invention also refers to binding through covalent forces. For example, antibodies suitably adsorbed to colloidal particles of the present invention include but are not limited to IgG or Fab fragment thereof; non-antibody proteins include but are not limited to fibrinogen, albumin and insulin. Non-protein molecules suitably include, e.g., lipopolysaccharides and lectins.

A variety of biomolecules can be conjugated to the shells of the colloidal particles. Antibodies or ligands conjugate to the gold shells similarly to the surfaces of the solid gold particles. Thus most antibodies or ligands are conjugated via procedures that utilize primarily hydrophobic interactions. For many antibodies and ligands this results in virtually irreversible binding of the protein (or the proteinaceous part of the molecule) to the gold surface but generally retains the binding properties of the conjugated protein. This methodology also works well for the solid gold and solid palladium particles. The gold shell increases the stability of the particles and permits the ready conjugation of antibodies or ligands via procedures developed by the inventors (Bleher et al., 2004, Microscopy and Microanalysis 10, suppl. 2: 158-159; Kaiser et al., 2007, Microscopy and Microanalysis, 13, suppl. 2:18-19; Bleher et al., 2008, J. Microscopy 230: 388-395), all of which are incorporated by reference. For rare protein targeting molecules, and for generally small ligands with high isoelectric points, which do not conjugate well, there are chemical crosslinking procedures that utilize various functional groups to crosslink the molecules or ligands to the particles, such as thiol cross linkers, for example dithiobis(succinimidyl propionate).

In some embodiments, the present invention provides for the production of colloidal nanobioparticles and colloidal nanobioparticle conjugates that can be targeted to specific cell types. In general, such colloidal particles include magnetic material surrounded by other material, which may or may not be magnetic. Thus, in one example, the colloidal particles include a “core” of magnetic material, which is surrounded by one or more “shells” of other material. The magnetic material that comprises the “core” of the colloidal particles can be made from a single element, e.g. magnetite or any other magnetic material. Alternatively, the magnetic material that comprises the “core” of the colloidal particles of the present invention can be made from a magnetic alloy. The shell may or may not be magnetic. Alternatively, if there is more than one shell surrounding the magnetic material, each shell may or may not be magnetic. In one example, the nanobioparticle conjugates include an iron (Fe) core and a gold (Au) shell (i.e., Fe/Au, core/shell). The shell may surround the core material in the form of one or more layers that cover the core. The layers may completely cover the core, or they may only partially cover the core.

The colloidal particles of the present invention are magnetic and can be inductively heated by an oscillating magnetic field. The degree of heating can be substantial; since a single 20 nm colloidal magnetite particle can generate up to about 8.3×10⁻¹⁸ Joules per hour (Kandela, 2006, Ph.D. Thesis, University of Wisconsin-Madison). This translates to temperatures of about 45° C. over ambient. The heat generated per particle for a given field frequency, strength, and time of exposure can be calculated by measuring the temperature increase of a known concentration of particles in a set volume of aqueous medium. In one example, measurement of heat production by core/shell particles is conducted in wells made of silicone (embedding mold) with small thermocouples running to the center of the liquid volume. The wells are placed in an oscillating magnetic field generator. The samples are placed in an air core solenoid where the field is generated and measured. This system allows for accurate measurements of small volumes. A distinct volume of cFeAu is placed in each well. Controls may include the same volume of water and equal numbers of colloidal gold particles placed in adjacent wells and simultaneously exposed to the same field parameters.

The size of such core/shell colloidal particles of the present invention can vary, for example, in the range of about 1 nm to about 500 nm, and is preferably in the range of about 5 nm to about 50 nm. The colloidal particles of the present invention are in one example spherical. However, the colloidal particles can also have other regular or irregular shapes, e.g. the colloidal particles can have shapes that are cuboidal, pyramidal, lobate, or geodesic, or combinations thereof. The particles can have smooth edges, ruffled edges, or combinations thereof. The sizes of these particles are usually categorized based on the size of smallest sphere they would fit into. For spherical or substantially spherical particles, the particle size refers to the particle diameter.

In one preferred example, the colloidal particles are spherical and they have about 10 nm diameter magnetite core. Various shell thicknesses can be used. For example, particles with gold shells of about 2.5 nm or about 7.5 nm are readily heated by the oscillating magnetic field. The advantages of the smaller shell is a smaller overall particle size while the thicker shell provides additional stability and a larger particle which can adsorb more targeting antibody molecules on its surface. More than one shell may be used, by overlaying multiple layers of same or different shell materials. When more than one shell layer is used, intermediate layers may be used to separate the shell layers. In some embodiments, the intermediate layers may include magnetic material as well. Thus, a variety of combinations of core and shell configurations, shapes, and sizes are possible.

The sizes indicated above are useful for in vitro and in vivo applications. If desired, preliminary studies may be conducted to determine the optimal size of particles for various applications. Based on initial results from such studies it may be useful to make adjustments to the core and shell thicknesses. As well, based on initial results from such studies it may be useful to make adjustments to parameters of the magnetic field used for inductively heating the corresponding particles. Thus, in some examples, an intermediate shell thickness of about 3.5 nm to about 4.5 nm may provide necessary particle stability with smaller overall particle size.

For example, particles can be synthesized as per Tables 1 and 2 below. Particles of different sizes can be made using, for example, the methods described in Meyer and Albrecht, 2002, Microscopy and Microanalysis 8 (Suppl 2); Meyer and Albrecht, 2003, Microscopy and Microanalysis 9 (Suppl. 2); Hillyer and Albrecht, 1999, Microscopy and Microanalysis 4: 481-490; Hillyer and Albrecht, 2000, Microscopy and Microanalysis 6 (Suppl. 2): 1006-1007; Hillyer and Albrecht, 2001, J. Pharmaceutical Sci. 12: 1927-1936, all of which are herein incorporated by reference. In some preferred embodiments, the water that is used in the synthesis of the nanobioparticles of this invention must be degassed (i.e. any gas must be removed). In some preferred embodiments, shell formation may be improved by partial surface oxidation of the core surface.

TABLE 1
Examples of synthesized colloidal particles with varying diameters

| | |
|---|---|
| Final concentrations [M] | Average diameter |

| Number (Sol) | Fe²⁺ | Fe³⁺ | of particles (nm) |
|---|---|---|---|
| 1 | 0.0025 | 0.005 | 4 |
| 2 | 0.0075 | 0.015 | 10 |
| 3 | 0.01 | 0.02 | 15 |
| 4 | 0.015 | 0.03 | 20 |
| 5 | 0.02 | 0.04 | 30 |

TABLE 2
Examples of synthesized colloidal particles
with shells of varying thickness

| | |
|---|---|
| Final concentrations | Gold shell |

| Number (Sol) | % HAuCl₄ | % NaBH₄ | thickness (nm) |
|---|---|---|---|
| 1 | 0.004 | 0.1 | 2.5 |
| 2 | 0.008 | 0.1 | 5.0 |
| 3 | 0.012 | 0.1 | 7.5 |
| 4 | 0.016 | 0.1 | 10.0 |

The colloidal particles of the present invention can optionally be conjugated to a variety of biomolecules, including but not limited to antibodies or antibody fragments as well as specific ligands, active fragments of ligands, glycoconjugates, cell surface-specific molecules, lipids, carbohydrates, drugs, labels, or any combinations thereof. In some embodiments, the conjugated agents can target certain cells or cell types, i.e. they can be cell-specific or cell type-specific. These particles can then be targeted to one or more specific cell types, i.e., the particles can be used to “label” specific cell types. Once the particles have been targeted to specific cells or cell types (i.e., have labeled those cells or cell types), targeted cell death can be achieved in as little as about 1.5 min to about 7 min by heating the target cell-adherent nanobioparticles in an oscillating magnetic field. For example, the methods of the present invention can be used to simultaneously label both cells in the primary tumor as well as any metastatic cells that have spread from the tumor. In addition, non-solid tumors like leukemias or lymphomas can be treated. The invention is especially useful for the treatment of tumors that metastasized, or for tumors that are not solid in nature, i.e. where only a relatively small number of tumor cells is collected together. Various aspects related to using the particles of the present invention as labels are described in Kandela et al., 2007, J. Histochem. Cytochem. 55: 983-990; Kandela and Albrecht, 2007, Scanning 29: 152-161; Kaiser et al., 2007, Microscopy and Microanalysis, 13 suppl. 2: 18-19; Albrecht and Meyer, 2008, In: Low Voltage Scanning Electron Microscopy, Chapter 5, Schatten and Pawley, eds. Springer Science, New York, N.Y., pp 171-196; Sims et al., 2006, In: Handbook of Biological Confocal Microscopy, Chapter 49, 3rd edition, Pawley, ed., Springer Science, New York, N.Y.; all of which are herein incorporated by reference.

In one example, the present invention provides conjugates, which include: a) colloidal particles that include a magnetic material and a shell that surrounds the magnetic material; and b) one or more substances that include biomolecules; where the substances are conjugated to the shells of the colloidal particles. The magnetic material may include iron, cobalt, zinc, or nickel, alloys of these, or any combinations thereof. The shells may include gold, silver, palladium, platinum, rhodium, molybdenum and ruthenium, alloys of these, or any combinations thereof. The shells of the conjugates may additionally include thermosensitive polymers, as described below. The colloidal particles may have a variety of shapes. Nonlimiting examples of suitable shapes include colloidal particles that are spherical, cuboidal, pyramidal, lobate, or geodesic, or any combinations of these. The sizes of the colloidal particles may range from about 0.5 nm to about 500 nm, and preferably from about 1 nm to about 100 nm. Various biomolecules may be conjugated to these colloidal particles. Nonlimiting examples of suitable biomolecules include proteins, peptides, antibodies, nucleic acids, antigens, glycoconjugates, cellular surface molecules, cellular components, ligands, toxins, or drugs.

The methods of the present invention are compatible with both in vitro and in vivo applications. The use of magnetic field for inductively heating of magnetic nanobioparticles avoids the development of resistance in the target cells since the active agent is physical, i.e., heat. Toxicity to the hosts is minimized. In one example of a preferred Fe/Au (core/shell) colloidal particle of the present invention, both elements are nontoxic and the magnetic field used to heat the particles is also non-injurious. Treatments can be repeated multiple times, thus minimizing and/or eliminating the target cell resistance and/or host toxicity. Treatments can be repeated with any desired frequency. Further, different numbers of particles can be used depending on the application. It is contemplated that the particles' size and level of heating are such that only target cells are killed; adjacent, untargeted or unlabeled, cells are typically not affected. If affected, the untargeted or unlabeled cells are only transiently affected, and they are not killed by the treatment. Only the targeted cells have sufficient label on their surfaces to be killed. In contrast to other approaches that use inductive heating of metal particles to produce bulk heating “cooking” of affected cells and tissue, the methods of this invention are used to only heat labeled cells. The amount of energy produced by the numbers and particle size of particles is only sufficient to heat local areas of the labeled cell membrane. Even adjacent cells in physical contact with labeled cells are not heated and are not damaged. Other cells may have occasional non-specifically attached particles but in extremely low numbers.

In some embodiments, the particles are sufficiently small, molecular in size, to readily access different body compartments. The magnetic properties of the particles permit their concentration in areas of interest by a standard, non-oscillating, magnetic field, which facilitates the specific labeling (targeting). These small particles provide contrast for imaging and their location can be seen by Computed Tomography (CT) scan and/or Magnetic Resonance Imaging (MRI) so that subsequently the oscillating magnetic field (used for heating the particles) can be applied either to the whole body or to specific sites where targeted cells are located. Use of dispersions of magneto-ionic particles as MRI contrast media is disclosed in U.S. Pat. No. 6,251,366, which is incorporated herein by reference.

Various in vitro and in vivo experimental studies where it is desirable to eliminate specific cell types will also benefit from the compositions and methods of the present invention. Elimination of unwanted or contaminating cell types in cell culture or among differentiating stem cells and elimination, in vitro, of host-reactive cells from bone marrow prior to transplantation, are also examples of potential applications.

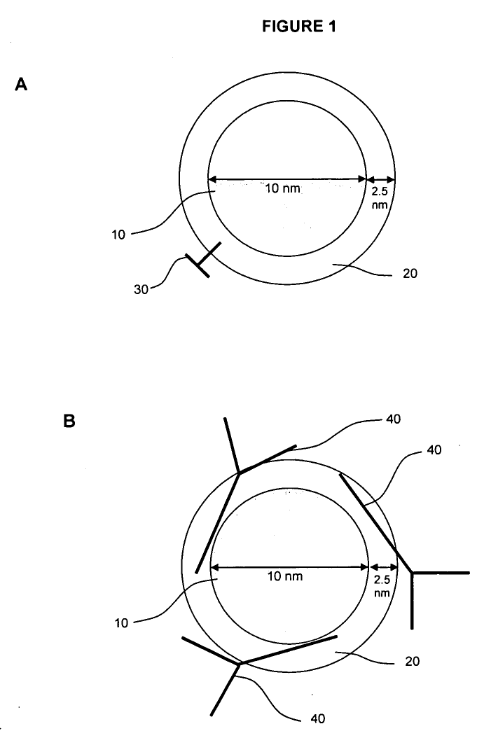

While magnetite particles can be effectively heated and can readily kill cells, they are not as stable as gold or palladium particles in a biological environment. The iron is substantially more reactive than the noble metals in a biological environment. Also the conjugation of antibody or ligand molecules to magnetite is not straightforward. In order to provide additional stability in a biological environment and to facilitate conjugation of specific antibodies or a variety of desired ligands while retaining full biological activity, the colloidal magnetic (nano)particles can be further coated with a gold shell, to generate magnetite/gold (core/shell) nanobioparticles, as shown in FIG. 1. In this example, the spherical colloidal particle is shown as a concentric sphere of colloidal iron/gold (cFe/Au) with a diameter of about 15 nm. The particle includes an iron core 10 with a diameter of about 10 nm, which is surrounded by a gold shell 20 (concentric in this example) with a thickness of approximately 2.5 nm, as shown in FIG. 1. The particle can optionally have one or more biomolecules 30 readily conjugated to it, as shown in FIG. 1A. As explained above, biomolecules may include one or more peptides, proteins, antibodies, antigens, nucleic acids, various biostructures or fragments thereof, and also may include cellular components, glycoconjugates, cellular surface molecules, any fragment(s) of any of these, or one or more therapeutic substances, e.g., drugs or other chemical substances intended for use in diagnosis, cure, treatment or prevention of a pathological condition, or any other ligands used in ligand-host interactions. In some preferred embodiments, the particle can have one or more antibodies 40 conjugated to it, as shown in FIG. 1B. The number of biomolecules attached to a colloidal magnetic particle may vary, and it may be as low as 1 or less (multiple particles attached to one biomolecule), and as high as 10,000, and is limited by the molecule size as it relates to the particle surface area and surface area of attached portion of each molecule. For example, for a relatively large particle of 100 nm, the surface area is 31,415 nm². Proteins conjugated to these particles are usually attached to the surface like somewhat squashed sausages or discs; typically, a little less than half of each protein's surface area is attached to the surface of the particle. For a typical molecule with a stokes radius of about 6 nm, half of the molecule's surface area is about 56 nm², so a maximum number of molecules is about that would fit on the surface is about 560. Attached molecules are smaller and larger and there are some considerations relative to end on vs. side on attachment. As well, some molecules (e.g. albumin) switch from one packing mode to the other depending on concentration.

More than one type of biomolecule may be attached to one particle, for example, one particle may include one or more antibodies and one or more peptides attached to it. The biomolecules may be attached to the particle via the shell (as illustrated in FIG. 1), via the core, or both via the shell and via the core. Biomolecules may also be attached to one another, to create linkages of two or more biomolecules. Such linked biomolecules may then be attached to the particles of this invention. Aspects of use of magnetic particles as substrates for immobilizing biomolecules such as ligands, which are suitable in the practice of the present invention, are disclosed, e.g., in U.S. Patent Application Publication No. US 2005/0201941 A1; and in U.S. Patent Application Publication No. US 2006/0233712 A1, both of which are incorporated herein by reference.

Colloidal metal particles according to one embodiment can be prepared using a variety of methods known in the art. For example, colloidal gold suspensions can be prepared by reducing gold chloride (HAuCl₄) with sodium citrate or sodium citrate and tannic acid to yield a 10-fold concentration (Albrecht et al. 1993, In: Immunocytochemistry: A Practical Approach, Beesley, ed., pp 151-176, Oxford University Press, Oxford, UK). To make concentrated colloidal gold suspensions, 2.5% maltodextrin can be added to the solution as a stabilizer prior to gold chloride reduction to prevent bead auto-aggregation.

The shells of the colloidal particles of the present invention can be made from a variety of materials. The materials include metals, such as gold, silver, palladium, platinum, rhodium, molybdenum, and ruthenium. A shell may be made of one type of material. Alternatively, a shell may be made of any number of materials, including alloys of materials. In one example, a shell can be made of gold. Gold has the advantage of being non-toxic and antibodies or ligands can be readily conjugated to it. However, shells made of silver, palladium or other metals can also serve a similar purpose. Aspects of methods useful for making these nanobioparticles can be found, e.g., in U.S. Pat. No. 6,699,507, which is incorporated herein by reference. The shapes of the shells can vary. In one example, the shell may be in the form of a concentric sphere, surrounding the magnetic core.

In one example, gold coated iron particles (cFe/Au) are synthesized via ammonium hydroxide oxidation of a mix of Fe³⁺ and Fe²⁺ iron salts in an aqueous medium. Varying the concentration of iron salts can be used to adjust particle size. To regulate the core size, salt concentration determines the number of nuclei that form initially under supersaturated conditions and then grow into nano-sized particles. The more nuclei the smaller the particles and the fewer the nuclei the bigger the particles because the total amount of metal is fixed.

The coating thickness can be determined, e.g., by the amount of gold added to the magnetite particles in the presence of a reducing agent, such as hydrazine. Increasing the amount of gold increases the thickness of the shells. It might be also possible to vary the time of the reaction. Hydrazine or borohydride are used as reducing agents for the shells because they don't result in cores or particles. Any reducing agent that also makes particles, for example citrate, would cause gold to accumulate as shells on the cores (e.g. iron) but it could also result in formation of entirely new nuclei and hence new particles entirely of gold. The hydrazine or borohydride seems only to facilitate the addition of shell material such as gold onto preexisting nuclei such as the surface of the iron particle and don't cause the formation of entirely new particles made only of the shell material). The gold shell does not affect the response to an oscillating magnetic field, thus heating of the core/shell particles occurs as for the cores alone, without the shell.

In one example, the present invention provides for the synthesis and characterization of colloidal magnetic particles in sizes from about 1 to about 100 nm, and preferably from about 4 nm to about 30 nm. The heating properties of these particles in oscillating magnetic fields of varying strength and frequency can be determined empirically.

The colloidal nanobioparticles of the present invention can be inductively heated. “Inductive heating” or “induction heating” is the process of heating a metal object by electromagnetic induction, where eddy currents are generated within the metal and resistance leads to Joule heating of the metal. In one example, an induction heater consists of an electromagnet, through which a high-frequency alternating current (AC) is passed. Alternatively, heat may also be generated by magnetic hysteresis losses. Often, iron and its alloys respond best to induction heating, due to their ferromagnetic nature. Eddy currents can, however, be generated in any conductor, and magnetic hysteresis can occur in any magnetic material.

The present invention provides for the use of oscillating magnetic field and magnetic nanobioparticles to kill cells. Targeting the colloidal particles to specific cell types, when used in conjunction with thermal heating, can be used to kill the targeted cells. An oscillating magnetic field (OMF) generator that is an air core solenoid version and operates in the range of about 100-500 kHz and up to about 0.008 Tesla, can be used to efficiently heat the magnetite particles of the present invention. It is possible to use various types of generators to generate an oscillating magnetic field suitable for the practice of this invention, for example using a toroidal core type of field generator. A toroidal core generator for generating oscillating magnetic fields is described in Dalessandro et al., 2006, IEEE Transactions on Power Electronics 21: 1167-1175, which is incorporated herein by reference. Other types of devices that can generate a magnetic field (e.g. oscillating magnetic field) that can heat the particles of these invention, may also be used. Different strength of magnetic field can be applied for varying periods of time and the corresponding degree of heating of the colloidal particles can be observed. Such empirical observations can be used to adjust the magnetic field strength and tailor it to the desired application.

The heat generated per particle for a given field frequency, strength, and time of exposure can be calculated by measuring the temperature increase of a known concentration of particles in a set volume of aqueous medium. In one example, measurement of heat production by core/shell particles is conducted in wells made of silicone (embedding mold) with small thermocouples running to the center of the liquid volume. The wells are placed in an oscillating magnetic field generator. The samples are placed in an air core solenoid where the field is generated and measured. The system allows performance of accurate measurements using small volumes. A distinct volume of cFe/Au particles is then placed in the well. Controls include the same volume of water and equal numbers of colloidal gold particles placed in adjacent wells all simultaneously exposed to the same field parameters.

Magnetic properties of the colloid are determined by the structure and the size of colloidal particles. For colloidal particles less than about 10 nm there may be only one domain available and thermal fluctuation will lead to an activation of re-magnetization processes. This is due to a decrease in the energy barrier for reorientation of the magnetic moment of particle with decreasing particle volume. An external oscillating magnetic field supplies the energy barrier. This energy dissipates as cFe moment relaxes to its equilibrium orientation (Chan et al., 1993, J. Magnetism and Magnetic Materials 122: 374-378). Measurements on the relationship between particle size, field frequency, and field strength as it relates to particle heating can be carried out, to establish the desired parameters for a variety of applications.

In one embodiment, the present invention makes use of colloidal magnetic particles (nanobioparticles) with conjugated biomolecules to serve as a means of both killing cells and delivering drugs. Various modifications are contemplated that could allow the present invention to target drugs otherwise unsuitable for systemic use to specific tissues, including tumors. In addition, certain properties of the particles themselves allow them to selectively kill target tissues even in the absence of drugs. Ingestion of these particles or particles coated with materials to facilitate ingestion by cells followed by exposure to an oscillating magnetic field leads to nearly complete cytotoxicity in a matter of a brief period of time, frequently in the matter of seconds. In contrast, the exposure to the magnetic field alone is not cytotoxic; neither is the ingestion of non-magnetic particles by cells. Exposure to the magnetic field further results in substantial intracellular heating. Intracellularly, the particles typically accumulate in vesicles or phagolysosomes. As such the particles are concentrated, more particles are present per cell, and the particles generate more localized heating than when they are attached over the surface of the cell. Hence cell destruction is more rapid and involves destruction of the vesicle membrane and hence of the vesicles themselves. This, in addition to the release of material from the vesicles and the particles from the vesicles, further disrupts cell function and may lead to cell death. The degree of heating can be readily modulated by adjusting the strength of the magnetic field and by the duration of exposure of the particles to the magnetic field, up to a limit until conduction away from the particle equals the heating capacity of the particle.

In some examples, the selective killing of various cell types is made possible through the use of cell-specific surface antigens, receptors, or receptor sites, which target particular cell molecules, particular cells, or particular cell types. Thus, the biomolecules may be specific for any desired target, such as a molecule, cell, cell type, tissue, tissue type, organ, organ type, etc. The biomolecules may attach to their respective targets directly, or via other, secondary biomolecules. Such secondary biomolecules may be present at the target, or they may be included as parts of the systems and methods of the present invention. The colloidal metal nanobioparticles can be conjugated to antibodies or ligands or active fragments of antibodies or ligands, as described in Albrecht et al., 1993, In: Immunocytochemistry: A Practical Approach, Oxford University Press, Oxford, pp 151-176, which is incorporated herein by reference. Some uses of particles of different shapes and compositions for labeling are described in Albrecht and Meyer, 2008, In: Molecular Labeling for Correlative Microscopy: LM, LVSEM, TEM, EF-TEM and HVEM, Ch 6, Biological Low Voltage Scanning Electron Microscopy, Schatten and Pawley (eds) Springer Science, New York, pp 171-196, which is incorporated herein by reference. In some embodiments, it is possible to use conjugates of colloidal metal nanoparticles of different elemental compositions or shapes (Meyer et al., 2006, Microsc. Microanal. 12, Suppl. 2: 32, which is incorporated herein by reference). Composite nanospheres and their conjugate with biomolecules are disclosed in U.S. Patent Application Publication No. 2005/0137334 A1, which is incorporated herein by reference. Bioprobes comprising magnetic nanoparticles are also disclosed in U.S. Patent Application Publication No. US2006/0142749 A1, which is incorporated herein by reference. Colloidal particles of different element composition for specific labeling purposes are disclosed in U.S. Pat. No. 6,699,507, which is incorporated herein by reference.

A variety of molecules can be conjugated to the magnetic nanobioparticles. Using the methods and the compositions of the present invention, in addition to conjugating “targeting” molecules to the particles (i.e. molecules that target the particles to a specific target—other molecule, cell, or cell type), it is also possible to attach therapeutic molecules (antibodies, proteins, non-proteinaceous molecules, drugs, nutraceuticals, etc.) to the particles (see, e.g., FIG. 1A), and to surround such particles with a heat-sensitive coating. The targeting molecules can thus deliver the particles to the desired cells (targets) and the heat-sensitive coating can be dissolved by inductive heating of the particle to release the therapeutic molecules. Thus the particle can serve the functions of both heat-induced cytotoxicity and drug delivery. Antibodies or antibody fragments suitably adsorbed to colloidal particles of the present invention include, but are not limited to, IgG or Fab fragment thereof; non-antibody proteins include, but are not limited to, fibrinogen (to produce binding and destruction of platelets and platelet thrombi which cause strokes and myocardial infarctions), albumin, and insulin. Non-proteinaceous molecules include, but are not limited to, lipopolysaccharides and lectins. In one example, the compositions and methods of the present invention can be used for tissue specific killing of particles conjugated to tumor-specific antibodies in normal vs. neuroblastoma tissue, for example in prostate.

The ability to selectively target and kill certain cell types may prove useful in various in vitro and in vivo applications. Many disease states such as tumors, virally infected cells, autoreactive cells, eukaryotic parasitic cells, and others rely on the specific suppression or removal of abnormal or unwanted cell types. Target cells cannot develop a resistance to the heated particles nor are the particles or the magnetic field injurious alone. It is also contemplated that the particles and the methods of the present invention can be used to target replicating vascular endothelial cells and destroy capillaries growing in tumors. The Fe/Au particles are also effective as labels in interference based LM, TEM and SEM, as well as effective contrast for MRI imaging enabling localization and identification of target cells prior to heating.

A variety of magnetic colloidal particles in the nanobioparticle size range from about 1 nm to about 100 nm, and preferably from about 5 nm to about 20 nm, is contemplated in the practice of the present invention. Smaller particles have greater mobility through a subject's body ands are thus more suitable for in vivo applications. The larger particles may become accumulated in the spleen, liver, or lymph nodes of the subjects to whom they are administered. However, the larger particles may preferably be used for in vitro work and for antimicrobial agents. These particles can be synthesized using methods known in the art. The particles can be heated by an oscillating magnetic field. For a variety of tested particles, effective heating occurs at approximately 500 kHz with field strength of up to about 0.01 Tesla, using an oscillating magnetic field apparatus that can be constructed using method known in the art, e.g. as described in Kandela, 2006, Ph.D. Thesis, University of Wisconsin-Madison, which is incorporated herein by reference. For example, the inventors constructed in house a magnetic generator with frequencies of about 100-500 kHz and a magnetic field strength of about 0.008 Tesla. The system consists of an oscilloscope V-200F (Hitachi, Japan), a signal generator (Wavetek 178, Electro-Biology, Inc., Parsippany, N.J.), a power amplifier (1040L power amplifier, Tucker, Garland, Tex.), capacitors (Sangamo type, Mansfield, Tex.), a solenoid coil wound in house, made from a number of coils wound of solid copper wire, silver coated copper wire, and from very small gauge copper tubing. For cooling of the copper wire (17 ga, 80 turns) wound core a water jacket was constructed that flows water in one end and out the other over the coils For the tubing variety the small gauge tube is wound around the core as the windings and water is run through the inside of the tubing to cool it. The cooling system was sufficient to prevent any coil heating or secondary heating of the samples). The core ID was about 4.5 cm, which was big enough for small tissue culture plates and mice. Suitable fixed and variable capacitors appropriate to the voltage were obtained through the University of Wisconsin High Energy Group at the Department of Physics. For example, to obtain an alternating magnetic field of 100-500 kHz, a signal generator serves as source of a sinusoidal waveform covering frequencies from 0.5 Hz to 50 MHz. The bias of a waveform can be adjusted with a DC offset voltage. The output power is usually limited by an internal source resistance of 50Ω (Ohms). The frequency and amplitude range can be selected with pushbuttons. Various combinations of fixed capacitors (capacitance: 1000 pF, 1800 pF, and 4000 pF) were connected to the power amplifier and to these a variable capacitor (capacitance: 0 to 1000 pF) was attached to obtain the magnetic field strength required. These functioned as resistors affecting the voltage that could be delivered to the solenoid to which they are attached. The magnetic flux in the coil was calculated using Coulomb's Law.

The particles of the present invention are administered so that they are delivered to the desired target molecules, target cells, cell types, and/or target tissues. Once the particles are delivered to the target(s), the particles can be subjected to an oscillating magnetic field, which causes heating of the particles. The degree of heating is substantial and can be in excess of about 45° C. over ambient. In one embodiment of the present invention, ingestion of these particles by cultured cells followed by exposure to the oscillating magnetic field leads to nearly 100% cytotoxicity in less than about 100 seconds of exposure to the oscillating magnetic field. In some embodiments of the invention, surface labeled cells are killed in 5 to 7 minutes to the oscillating magnetic field, depending on the extent of labeling. Exposure to the magnetic field alone or ingestion of non-magnetic colloidal particles of the same size and concentration followed by exposure to the oscillating magnetic field does not produce the cytotoxic effect nor do the magnetic particles themselves have a cytotoxic effect. The frequency and strength of the oscillating magnetic field is harmless. Thus, biomolecules conjugated to magnetic colloidal nanobioparticles can be used in vivo or in vitro to selectively injure or kill (remove) targeted cells (eukaryotic or prokaryotic). This can be useful in a variety of applications where it is desirable to damage or kill cells identifiable using specific cell surface antigens or specific ligand receptors. It is contemplated that the particles and methods of the present invention can be used to specifically target and kill cells, including, but not limited to, tumors, autoreactive cells, virus-infected cells, activated platelets, etc. For example, cAu nanoparticles are absorbed in various organs including the brain. The amount of absorption decreases generally with increasing nanoparticle size. Nanobioparticles that are about 4 nm or smaller can thus be used to kill specific cells in the brain and/or to deliver molecules to the brain. Colloidal iron particles are approved by the FDA for treatment of iron deficiencies and are non-toxic.

In some examples, particles can be targeted to the area of interest via the use of standard fixed magnetic fields. Once in the area, particles diffuse to specific targets and attach to specific cells as determined by the antibody or ligand conjugated to the particle. Once they have reached the specific targets the particles are exposed to the oscillating magnetic field which heats the particles. Cells are damaged or killed by the thermal heating. The degree of heating is dependent on particle size, length of exposure to the field, strength of the field, and the oscillation frequency of the field. Only particles exposed to the oscillating field are heated so that particles accumulating elsewhere via nonspecific mechanisms are not heated and exert no effect.

In one embodiment, the magnetic colloidal nanobioparticles can be coated with one or more thermosensitive polymers. A “thermosensitive polymer” refers to a polymer that can readily be dissolved upon exposure to change in temperature, and in particular upon heating. These thermosensitive polymers can be made up of hydrophobic, thermosensitive parts, and hydrophilic parts. Examples of thermosensitive polymers are shown in International Patent Application Publication No. WO/2003/101486, which is incorporated herein by reference. In one example, the thermosensitive polymers that are used to coat the magnetic particle have LCST (low critical solution temperature) about 4° C. to 10° C. above ambient physiological temperatures. The polymers shrink and release entrapped molecular species at the LCST. This permits coating of the magnetic nanobioparticles with a thin (e.g. about 2.5 nm or more) coatings of polymer plus targeting biomolecules such as antibodies or ligands. As well, active drug species can be incorporated in the thermosensitive polymer. One or more coatings of thermosensitive polymers can be applied. Biomolecules can be embedded into these polymers, can be conjugated to the surface of the polymers, or they can be both embedded and conjugated. Once targeted to particular cells or internalized/endocytosed, the particles are heated resulting in the polymers releasing the biomolecules. The thermosensitive polymers can be biodegradable. Biocompatible coatings suitable for magnetic nanoparticles are disclosed, e.g., in U.S. Pat. No. 7,074,175 B2, which is incorporated herein by reference.

Administration of the particles can be performed in a variety of ways. Particles of these sub-molecular and molecular size ranges distribute widely throughout the body and can be administered by intravenous (IV) and intraperitoneal (IP) routes, and are absorbed if given via the oral route. Large sized particles, above 20 nm, when not specifically targeted, tend to eventually accumulate nonspecifically at sites of phagocytosis such as the spleen, liver, kidney, or lymph nodes. The smaller particles, if not specifically targeted, tend to be more evenly distributed throughout the body.

Once targeted to the cell surface and there attached to the target cell or internalized/endocytosed, the particles are heated about 4° C. to 10° C. via the application of the oscillating magnetic field and the polymer releases the drug directly on the surface or in the cytoplasm of a target cell. Drug is released only where the oscillating magnetic field is applied so that drug is not released by particles which accumulate elsewhere by non-specific mechanisms. Since the heating required is minimal, cells need not be damaged by the heating so cytotoxic compounds or beneficial compounds, such as antibiotics, can be released specifically at targeted sites without concern for thermal damage to cells and tissues. In some cases, however a combination of thermal damage and cytotoxic compound release may be desirable and is possible. This system allows for the release of high concentrations of active compound directly to targeted cells while overall (whole body or whole system) levels remain low. Thus drugs not useful or that cannot be used at high whole body concentrations can be effectively employed in the magnetic nanobioparticle targeting system. Active compounds with short half lives also can be used more effectively because they are released at or very close to the site of action. In addition, due to the selective nature of using the nanobioparticles, the different mechanism of action from existing chemical approaches, and absence of side effects, the system can, if desired, be used in conjunction with other therapeutic approaches without increasing the level of side effects such as general host toxicity.

Optimal conditions for inducing targeted cell death can vary depending on effects of nanobioparticle size and numbers and magnetic field strength and frequency on target cell killing as well as the particular target cell type. Relative sensitivity related to different cell populations is similar but exact conditions are determined empirically. Both cell lines and primary cell cultures obtained from live animals can be utilized for practicing the methods of this invention. For example, determination of the efficacy in killing can involve 3 primary Fe/Au nanobioparticle sizes in concentrations of about 20, about 200, and about 1000 particles per cell. Numbers per cell are varied by varying the concentration of added particles. In one example, the magnetic field parameters include field strength of about 0.008 Tesla and an oscillation frequency of about 500 kHz. Initial exposure times are about 3 min. Based on the findings at about 3 minutes, shorter exposure times (e.g. about 30 seconds, about 60 seconds, and about 90 seconds) or longer exposure times (e.g. about 5 minutes, about 10 minutes, about 15 minutes, about 20 minutes, about 30 minutes, etc.) may also be employed. Field strength and oscillation frequency may be modified to be optimal for each particle size.

In addition to in vitro and in vivo cell separation, cell killing, or targeted in vitro and in vivo drug release, there are a number of other potential uses for this system. Specific ablation of specific cell types in developmental studies (e.g., embryogenesis) may be facilitated. Heating particles (below cytotoxic levels) that are attached to cells appears to increase their uptake such that intracellular delivery of nucleic acids, or a variety of other pharmacologically active agents, tracking dyes, etc. may be more effectively delivered into targeted cells. The particles of the present invention are typically readily contrasted in MRI imaging. This may prove useful in imaging the specific areas where targeting occurs to confirm particle location or to identify sites of nonspecific accumulation.

The present invention contemplates the use of the compositions and methods described herein to destroy thrombi. In one embodiment, the particles are coupled to fibrinogen to label activated platelets. Platelets, or thrombocytes, refer to the cells circulating in the blood that are involved in the cellular mechanisms of primary hemostasis leading to the formation of blood clots. Activated platelets are the ones in clots and they express the fibrinogen receptor on their surfaces, whereas normal, unactivated platelets do not. Thus, the present invention can be used to destroy the membrane of the activated platelets as per any other cell. Current therapy for thrombi causing strokes and heart attacks is imperfect and must be used very early after a clot forms in a vessel. Using the heated nanobioparticles alone or with other current therapy may be a significant improvement for destroying thrombi.

The present invention also contemplates the use of the compositions and methods described herein for thermELISA applications. The thermELISA can use the antibody attached core/shell magnetite particles of the present invention instead of the standard antibody with enzyme. The samples can last for long periods of time and do not have to be run immediately and can be archived for subsequent analysis without loss of activity. An oscillating field can be applied and the differences in the increase in temperature in the different wells (e.g. measured by a sensitive laser thermometer) would be proportional to the amount of antibody. To achieve higher sensitivity, heating for longer times could be used.

In general, the methods of the present invention have applicability to human systems, to animal systems, to plant systems, to prokaryotic systems, as well as to non-biological systems where it is desirable to release a compound at a specific location. In such instances, these carrier nanobioparticles can be incorporated into flow systems or cell sorting systems where it is desirable to release a compound, dye, or toxic agent at a particular spot in the flow. This can be accomplished simply by including the particles in the fluid phase and applying the proper oscillating magnetic field at the point needed. Sensor and electronic applications, where extremely localized (nanoscale) heating or release of active agent is required, are also possible.

Colloidal particles in the small size range of about 1 nm to about 50 nm can be absorbed via the intestinal tract and can be detected in substantial amount in various organs including, e.g., the brain, liver, spleen, kidney, and visualized with a microscope. Thus, the invention provides a delivery system for biomolecules or drugs to an in vivo target, which includes providing the colloidal particles of the present invention in particle sizes from about 1 nm to about 80 nm, preferably about 1 nm to about 50 nm, as carriers for biomolecules or drugs. Particles of shapes differing from the spherical cAu particles should also have value in the delivery system in accordance with the present invention, due to increased surface area and increased potential for binding, or as an indication of the presence of several specific labels or drug carriers at a given location.

In one embodiment, the present invention provides methods of selective introduction of compounds or small particles into targeted cells that are sub-lethally damaged. The particles of this invention can be directly conjugated to specific targeting antibodies or ligands. The specific antibodies or ligands target the particles to specific individual cells of interest. Once targeting is complete, an oscillating magnetic field is applied only to the target area in order to heat the particles up to 45° C. over ambient temperature. This produces irreversible thermal damage to membranes of the targeted cells or microorganisms. Lesser heating times produce repairable membrane holes such that the entry of biologically active agents is facilitated through the small holes which the cell then repairs. Thus, via short time exposure of the magnetite nanobioparticle labeled cells, it is possible to use sub-cytotoxic damage as a means to transport biologically active molecules into cells. Cells can be co-incubated with gold or palladium (non-magnetic and hence non heatable) nanobioparticles of sizes from 3 nm to 50 nm that have been conjugated to albumin. The level and location of gold or palladium in the cells can be determined by backscattered SEM labeling and Energy Filtering TEM imaging after the cells have repaired the membrane damage. This makes it possible to see what sizes of nanobioparticles can get into the cells that are compatible with membrane repair and cell survival.

Preferably, the biomolecules sought to be delivered include nucleic acids, proteins or peptides, e.g., an antibody or an antigen. Such metal colloid conjugates are prepared by using aspects of known methods, e.g., as described in Albrecht et al., 1993, In: Immunocytochemistry: A Practical Approach, Beesley ed., Oxford University Press, pp. 151-176; and also in U.S. Pat. No. 5,384,165, all of which are incorporated herein by reference. For example, cAu can be conjugated to virtually any protein by hydrophobic bonding under conditions by which the conjugates retain the properties of the protein. Use of colloidal particles of small size compared to the ligand, e.g., protein, antibody, ensures that the conjugation does not affect the activity or function of the protein.

Experiments with cell lines exemplified herein reflect the cell types that can be used for in vivo labeling and killing studies. These cell types include any cell type that can be specifically or fairly specifically labeled on its surface by a molecule such as an antibody or ligand. Examples of cell lines that have been tested include lines obtained from the American Type Culture Collection (ATCC), Manassas, Va.: (i) RAW 264.7, which is a macrophage cell line from murine (antigen Mac-1); (ii) 2025, which is a human prostate carcinoma line (antigen PAP); (iii) 22Rv1, which is a human prostate carcinoma line (antigens PSMA, PAP); (iv) Raji, which is a human B cell line, i.e. Burkitt lymphoma (antigens CD19, CD20, B220); (v) HCC 11954, which is human breast carcinoma line (antigens C-erbB2, her2/neu); (vi) OvCar 3, which is a human ovarian carcinoma cell line (antigen muc16) and (vii) SK-MEL-5, which is a human melanoma line (antigen HMB-45). Additional examples of cell lines suitable for the practice of this invention include activated platelets, yeast cells, and SV-40 infected cells. In one example, for studies on the cytotoxic ability of the inductively heated Fe/Au, core/shell nanobioparticles, a prostate cancer cell line expressing PSA, prostate specific antigen, was cultured in vitro alone or with a normal, PSA negative, prostate cell line. The cells were labeled with primary anti-PSA antibody followed by secondary antibody coupled with either Fe/Au core/shell particles or just solid Au (nonmagnetic and hence not heated) particles of the same size. Labeled and unlabeled cells were exposed to an oscillating magnetic field for various times up to about 7 minutes.

If desired, surface adherent and any internalized particles can be visualized by light microscopy, e.g., directly by interference based microscopy and secondarily by fluorescence, and alternatively by scanning electron microscopy using secondary electron imaging and high resolution backscattered electron imaging. Examination by SEM and TEM provides high resolution images of cell structure and the extent of cell damage. Au and Pd nanoparticles or nanobioparticles can serve as non-magnetic control particles. Macrophage cell lines can be used for internalization of particles while tumor cell lines and virally transformed cells can be used for the surface labeling and cell killing applications. Cytotoxicity can be determined by Sytox green and Annexin V assays following exposure of labeled cells to the magnetic field. Cells can also be examined by bright field, phase contrast, and interference based (DIC and AIC) light microscopy.

The colloidal nanobioparticles in accordance with the present invention are generally compatible with existing preparative procedures for chemical or physical (cryo)fixation, including staining, solvent dehydration, polymer, embedding, thin sectioning, and dehydration by the critical point or freeze drying procedures. The colloidal nanobioparticles of the present invention in multiple therapeutic procedures can be utilized for their differing particle shapes or differing elemental composition or both.

An important aspect of the systems and method of the present invention is the process by which cells, labeled with the core/shell particles, are killed or damaged. This involves only localized heating of the cell or microbe membrane to which the particle is attached. It does not involve bulk heating of tissue or even heating of the entire cell. Given the particle size and number of particles per cell, and the heat generated per particle, it is impossible to heat the surrounding tissue or even to heat the labeled cell to any significant amount other than to damage the membrane in immediate contact with the core/shell particles. This is important for several reasons. First, only the cells labeled with nanobioparticles are damaged. Cells adjacent to and even in contact with the labeled cells are not heated and hence are not damaged and collateral damage is limited. In contrast, in systems using typical chemotherapeutic agents or radiation the agent generally affects target cells as well as surrounding tissue. With cytotoxic chemotherapeutic agents, all cells in the body are exposed to the agent. Multiple dangerous side effects can occur which limit the concentration of chemotherapeutic that can be used. In addition, the present invention is in contrast to other approaches to kill tumors, where large iron particles are directly injected into tumors and no provision has been made to selectively label tumor cells since the objective has typically been to cause the accumulation of large amounts of iron in and around the tumor area and then heat the particles to such an extent that all the cells in the entire area are basically “cooked” without any selectivity. Thus, when treating tumors, the methods of this invention use the conjugated core/shell particles so that only the tumor cells are labeled and killed. Thus cells in both primary tumor as well as close and distant metastases are labeled and killed but adjacent, unlabeled cells are retained. Hence with spreading brain tumors or other invasive tumors, large areas of normal cells do not have to be killed or surgically removed in order to destroy all the tumor cells. Heavy doses of toxic chemotherapeutic agents do not have to be applied. Furthermore tumors such as leukemias or lymphomas, autoreactive immune cells, or microorganisms that are often not contiguous or concentrated in any one place and which often live in the blood stream, bone marrow, or in the lymph fluid can be selectively labeled and killed with no or only minimal damage to any other cell types.

In one aspect, the amount of metal (e.g. iron and gold) injected with the specific labeling system is also far less than required for any bulk heating process. The number of particles needed and hence the mass necessary for a given application can be determined by an estimate of the total number of target cells to be labeled and the number of antigenic (e.g. antibody or ligand) binding sites per cell. While in most instances this would involve informed estimates, the total amount of conjugated core/shell administered would be an order of magnitude or possibly several magnitudes less than the amount of iron needed for bulk heating of a solid tumor tissue.

Another aspect of the selective nature of the methods described herein involves the limited heating of labeled target cell membranes. In this case cells develop small holes which they can repair and hence remain alive and functional. The cells in which these holes develop can be selectively targeted. The development of the holes allows larger particles or molecules, which ordinarily could not enter the cell to enter the cell prior to repair of the holes. So, for example, in the case of genetic material, specific cells or cell types, rather than all cell types, can be targeted to receive the genes or gene fragments. Unlabeled and hence unheated cells, even cells adjacent to targeted cells, would not develop the small holes and the genetic material could not enter these cells.

The process of transferring material into cells with partially damaged membranes would be effective when applied in vivo but would also be useful in in vitro applications as well where transfection of genetic material to selective cell types, rather than all cells in a culture, is desirable. In other applications, bone marrow cells can be targeted, or undifferentiated stem cells can be targeted for removal from a differentiated population prior to transplant. Since the core/shell conjugates are similar in size and solubility to antibody molecules the application in vitro is straightforward and labels diffuse throughout the media or buffer in a manner identical to any other protein of similar size.

### EXAMPLES

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

Synthesis and characterization of magnetite/gold (Fe/Au) core/shell particles and gold (Au) and palladium (Pd) control particles. In one example, two sizes, about 4 nm and about 10 nm, of core particles were synthesized. A shell with about 2 nm thickness was used with the about 4 nm core, thereby producing an about 8 nm overall particle size (i.e., spherical particle with about 8 nm diameter). In some examples, the about 10 nm cores were combined either with an about 2.5 nm shell or with an about 7.5 nm shell to provide spherical particles of about 15 nm and about 25 nm overall diameter.

In order to generate the required oscillating magnetic field of about 500 kHz at about 0.008 Tesla, an air core solenoid was constructed. The solenoid could accommodate small samples including about 3.5 cm tissue culture dishes or mice.

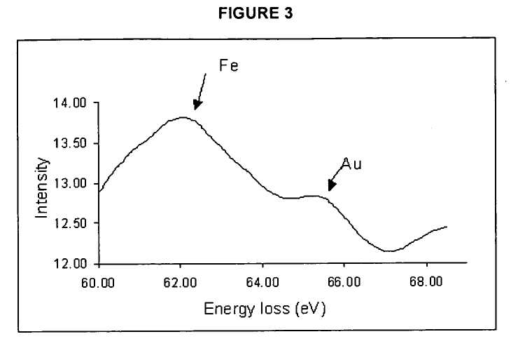

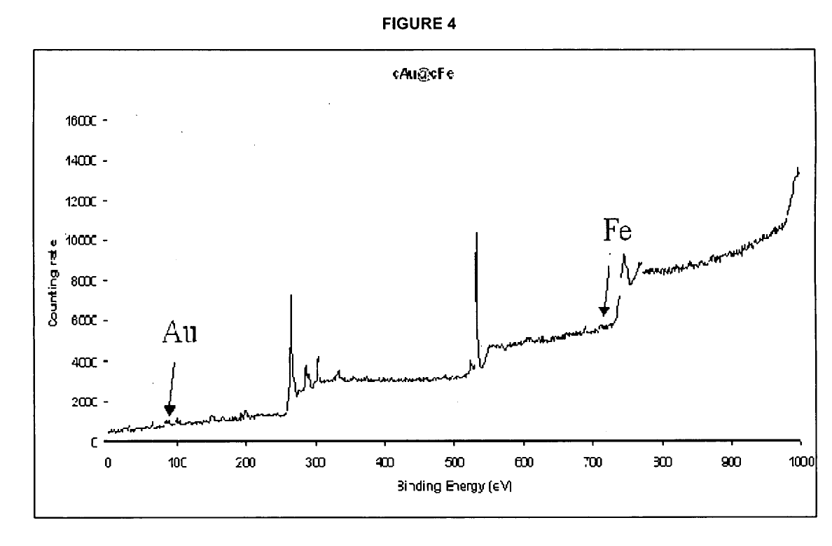

The gold coated iron particles were observed and characterized in a variety of ways, using: electron spectroscopic imaging (ESI); transmission electron microscopy (TEM); UV-Vis spectroscopy; parallel electron energy loss spectroscopy (PEELS), as shown in FIG. 3; and X-ray photoelectron spectroscopy (XPS), as shown in FIG. 4. ESI analysis confirmed the composition of the core shell. The gold shell on the iron cores was easily observed in the obtained ESI images.

Determination of the number of particles bound to cells. To achieve desired effects, e.g. killing of cells, it may be important that the cell-specific biomolecules (e.g. antibodies) bind in sufficient numbers specifically to the target cell populations. In one example, about 200 particles of the about 10 nm Fe core size can kill targeted cells in as little as about 90 seconds to about 420 seconds. Generally there are thousands to tens of thousands of target molecules on the target cell surface and thus the number of antibodies or other conjugated molecules could be substantial.

To visualize the distribution of particles cultured cells are labeled with primary antibody conjugated to particles for about 5 min at a concentration of about 1×10¹³ particles per ml. This concentration results in maximal labeling within several (about 5) minutes. Cells are washed to remove unbound particle conjugates and then labeled with a second antibody which is conjugated to a fluorescent compound, Alexa dye. Fluorescence light microscopy is used to determine distribution of label on the living cells (Kandela et al., 2004, Microscopy and Microanalysis 10, Suppl. 2; Kandela et al., 2005, Microscopy and Microanalysis 11, Suppl. 2). Alternatively, labeling with unconjugated primary antibody followed by second antibody nanobioparticle conjugates can be employed in cases where primary antibody is expensive or in short supply.

Numbers of particles actually bound to cells were determined both by correlative fluorescence imaging and by high resolution field emission SEM of fixed, critical point dried cells. Antibody-conjugated nanobioparticles are estimated quantitatively using an anti-isotype secondary fluorescence conjugated antibody and analyzed by flow cytometry. Short labeling times are preferably used to ensure that the majority of the particles remain on the cell surface. Following labeling, cell cultures are lightly fixed in 2% paraformaldehyde before being incubated with saturating concentrations of appropriate anti-isotype secondary antibody. Adherent cells are lifted from coverslips using a cell scraper. The degree of nanobioparticle labeling is determined by flow cytometric analysis using a FACSCalibur flow cytometer (BD Bioscience).

For SEM analysis, substrate adherent cells are fixed and dried by the critical point procedure for examination in a Field Emission SEM in the secondary and backscattered electron modes so that individual particles can be observed and counted on the cell surfaces. Once initial binding levels are determined, changes in concentration of the primary antibody-particle conjugate and length of the initial labeling can be modified to provide different levels of labeling ranging from tens, hundreds, and thousands of surface labels.

Killing of cells using the synthesized Fe/Au (core/shell) particles. The methods of the present invention were successfully used with the prostate cancer cell line ATCC #2505 and anti-human prostate acid phosphatase conjugated to the Fe/Au (core/shell) particles. In some examples, both Fe/Au particles and solid gold (non-magnetic) particles of the same size were conjugated to anti-prostate specific antigen antibody and used to label prostate cancer cells in vitro. Both particle types were shown to label cells which expressed prostate-specific antigen on their surfaces. No labeling was seen when control antibody was conjugated to the Fe/Au or the Au particles. When the labeled cells were exposed to an oscillating magnetic field only the cells labeled with Fe/Au particles were killed.

Micrographic images of particles, obtained using ESI (electron spectroscopic imaging), were used to image and define the location of gold and iron relative to the gold-magnetite, core/shell nanobioparticles. More specifically, magnetite-gold, core/shell colloidal nanobioparticle samples (7 μL) were placed on formvar coated Ni 600 mesh grids. The gold coated magnetite nanobioparticles were examined in a Zeiss EFTEM 912 with a Lanthanum Hexaboride filament and an emission current of 12 μA with 120 kV acceleration voltage. Gold- and iron-specific signals were identified using Electron Spectroscopic Imaging (ESI) by applying the 3-window method at energy losses of 75, 45 and 35 eV for the Au_{02,3}-ionization edge and 62, 50 and 35 eV for the Fe_{M2,3}-specific ionization edge. For both gold and iron, exposure time was two seconds at an illumination angle of 1.25 mrad. Energy filtered images were analyzed by the power law method using ESI Vision software from SIS and images of 1024×1024 pixels were taken with a slow scan CCD camera (SIS, Muenster, Germany). Loss electrons were used to produce images. Zero loss images of structure can be directly compared to image produced with loss electrons, to identify the location of specific elements within the samples.

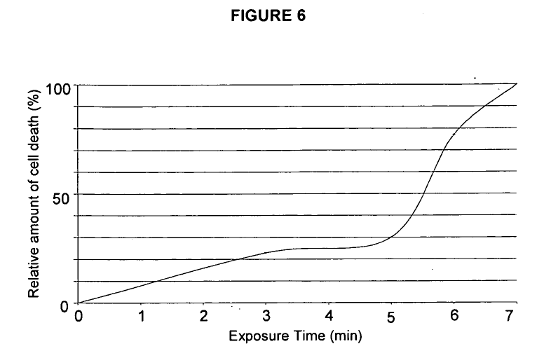

FIG. 6 is a graph showing the percent tumor cell death vs. time of exposure to the oscillating magnetic field. The abscissa shows the time of exposure to the magnetic field (min). The y-axis shows the relative amount of dead cells (%).

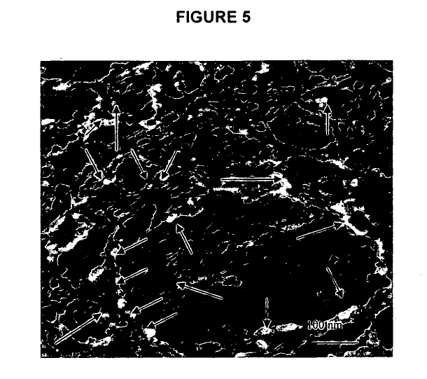

No cytotoxicity was seen with co-cultured, unlabeled, normal prostate cells or with tumor cells labeled with the antibody-Au conjugates. Cells labeled with the core/shell particles but not exposed to the magnetic field were also not killed. However, the cells labeled with the antibody conjugated Fe/Au core/shell particles began to exhibit cytotoxicity by about 3 to 4 minutes and by about 7 minutes cytotoxicity was about 100% (Table 3, FIG. 6). Since the volume of the surface membrane attached nanobioparticles was roughly about 1/250,000 of the total cell volume, general heating of the cell could not account for cell death. SEM images of cell membranes demonstrated observation a progressive “melting” of membrane near heated particles. This resulted in the formation of multiple membrane holes which increased in size with increasing length of exposure (FIG. 5). Not wanting to be bound by the following explanation, it is possible that cell death occurs due to increased membrane permeability, i.e., “melting” of the plasma membrane (Kaiser et al., 2007, Microscopy and Microanalysis, 13 suppl. 2: 18-19). The membrane bound magnetite particles probably heat the cell membrane and produce holes which enlarge over time. Once a certain size or porosity of the membrane is reached the cell cannot repair the damage. This is supported by data showing that as the magnetic field is applied small holes begin to appear but if the magnetic field exposure is terminated at this point the cells can repair the damage and do not die. When the cells are exposed for several minutes to a magnetic field of sufficient intensity, many cell membranes suffer extensive damage and numerous larger holes in the cell membrane become visible.

Table 3 shows data obtained using the prostate cell line. The percent of non-viable cells per treatment group are indicated. Cell toxicity was determined with a Cell Titer Blue assay.

TABLE 3
Relative amounts (%) of non-viable cells per treatment group

| | | % Dead not | % Dead | Total |
|---|---|---|---|---|
| Sample | % Alive | fluorescing | round | % dead |
| 0 minutes-Gold | 100 | 0 | 0 | 0 |
| 7 minutes-Gold | 100 | 0 | 0 | 0 |
| 0 minutes-Gold- | 100 | 0 | 0 | 0 |
| coated magnetite | | | | |
| 3 minutes-Gold- | 77 | 9 | 14 | 23 |
| coated magnetite | | | | |
| 5 minutes-Gold- | 70 | 5 | 25 | 30 |
| coated magnetite | | | | |
| 6 minutes-Gold- | 23 | 69 | 8 | 77 |
| coated magnetite | | | | |
| 7 minutes-Gold- | 0 | 87 | 13 | 100 |
| coated magnetite | | | | |

Both magnetite and magnetite core/shell particles were tested with two cell lines, a macrophage cell line where particles are internalized and a prostate tumor cell line where antibody conjugated Fe/Au core/shell particles are adherent to specific cell surface antigens. Cytotoxic potential (Table 4) and the ability to visualize these particles in SEM and TEM were examined.

Trypan blue staining of nanobioparticles labeled cells demonstrated that cells with no exposure to the oscillating magnetic field remained alive. Cells exposed to the field for 60 seconds were blue, indicating cell death.

Micrographs of CRL 2505 prostate cell line were obtained, using bright field microscopy and fluorescence light microscopy. CRL 2505 cells were labeled with 25 nm Fe/Au nanobioparticles and exposed to 500 kHz oscillating magnetic field at about 0.008 Tesla for about 7.5 minutes. Sytox green staining showed substantial cell killing. The control cells were labeled with about 25 nm Au only and exposed to the same field for the same length of time. No cell killing was seen.

Labels on the tumor cell surface were visualized using SEM. Control cells had about 25 nm Au only (non-magnetic) particles attached to the anti-tumor cell antibody, and were exposed to the magnetic field. Other control cells had not been exposed to the magnetic field while. Both types of control cells were undamaged. Cells that were labeled with about 25 nm Fe/Au particles, but were not exposed to the magnetic field, were not damaged. Cells that were labeled with about 25 nm Fe/Au particles, and were exposed to the magnetic field, showed substantial damage to the cell membrane and were dead.

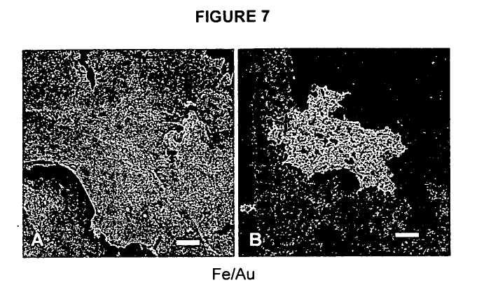

FIG. 7 shows lower magnification SEM images (micrographs) of Fe/Au labeled cells. The cell shown in the left image (FIG. 7A) has not been exposed to the magnetic field, whereas the cell shown in the right image (FIG. 7A) was exposed to the magnetic field. The right cell (FIG. 7B) shows substantial damage and cell debris remains.

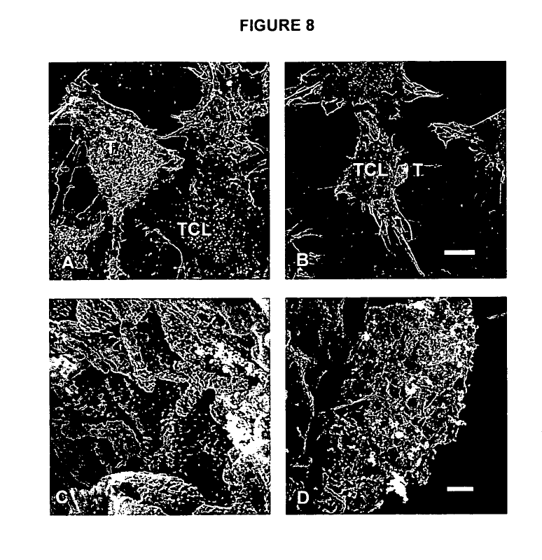

FIG. 8 shows both lower (A, B) and higher (C, D) magnification SEM images (micrographs) of mixed co-cultured normal prostate cells (TCL) and prostate cancer cells (T). In the lower magnification panels (A, B) the scale bar represents 5 μm. In the higher magnification panels (C, D) the scale bar represents 200 nm. The cells were labeled by rabbit anti-PSA IgG, and then further labeled with donkey anti-rabbit secondary IgG conjugated to cFeAu. Various controls were also labeled with donkey anti-rabbit secondary IgG, or with secondary IgG conjugated to cAu₁₅. In contrast to the left images (A, C), the samples shown on the right (B, D) were exposed to an oscillating magnetic field (OMF) with frequency of about 500 kHz at about 0.008 T for about 7.5 minutes. While normal cells were unaffected, cancer cells labeled with cFeAu nanobioparticles were selectively destroyed after exposure to an OMF. In FIGS. 8B and 8D, the remainders of a destroyed cancer cell between unaffected normal cells are shown.

TABLE 4
Cytotoxic potential of various colloidal particles
used for treatment of phagocytic macrophages

| Type of treatment, with/without oscillating magnetic field | |
|---|---|
| (OMF) for 60 seconds (400 Hz at 0.008 Tesla) | % of cells dead |
| cFe, with OMF | 85.3% |
| cFe, without OMF | 3.4% |
| cAu, with OMF | 4.3% |
| cAu, without OMF | 3.2% |
| No nanobioparticles, without OMF | 2.5% |
| no nanobioparticles, with OMF | 3.0% |

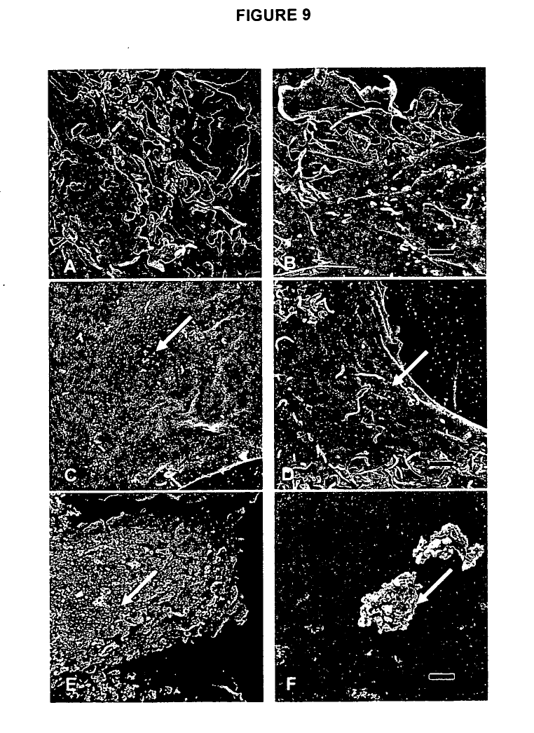

Selective killing of SV-40 infected cells. FIG. 9 shows SEM micrographs of normal prostate cells (YPEN-1). In contrast to the left images, the samples shown on the right were exposed to an oscillating magnetic field with a frequency of 500 kHz at 0.008 T for 7.5 minutes. The cells were labeled by mouse anti SV 40 IgG then further labeled with donkey anti mouse secondary IgG (A and B), secondary IgG conjugated to cAu₁₅ (C and D) and secondary IgG conjugated to cFeAu (E and F). Images were taken with a backscattered electron detector; metal nanobioparticles appear as bright spheres (arrows). Control cells, treated with buffer or cAu did not show any cytotoxicity, even after exposure to OMF. Control cells which were labeled with cFeAu but not exposed to an OMF showed no cytotoxicity. In contrast, cells labeled with anti SV-40-cFeAu, followed by OMF for 7.5 minutes were killed. Membrane integrity of this cell was severely compromised (F). Scale bar represents 500 nm.

Selective killing of yeast-infected cells. In some examples, selective killing of yeast cells was performed as follows.

Candida albicans culture techniques. The yeast cells were cultured from a sample provided from the Department of Medical Microbiology and Immunology at the UW-Madison, which was grown in test tubes with a broth solution. Hemacytometers were used to count the yeast cells, and 0.2% Trypan blue indicator dye was used to determine the viability of the cells.

Synthesis of core/shell nanobioparticles. Colloidal iron (cFe) particles were synthesized with FeCl₂, FeCl₃, H₂O, and NH₄OH. The FeCl₂ and FeCl₃ was weighed in appropriate ratios and mixed in a water solution. Argon gas was used as an unreactive atmosphere for the reaction. Next, 8 M NH₄OH was added to the mixture in small increments, with constant stirring. Once all of the NH₄OH was added, the mixture was heated at 80° C. for approximately 30 minutes. The argon gas, bubbled through the reaction mixture, produced sufficient turbulence to constantly mix the ingredients for the elapsed time. Finally, the precipitate was concentrated, rinsed with distilled and deionized H₂O, and stored in a water solution.

Gold coating process. The iron particles were then completely coated with gold. The cFe described above was used for the core, while 5.5% Hydrazine and 4% HAuCl₄ were used to produce the shell. Double distilled H₂O was added to a flask, and argon gas was bubbled through the water. The gas remained in the flask until the completion of the reaction. The water was stirred at a slow pace using a plastic stir rod as the stock solution of cFe was added. The stirring rate was then increased, and the Hydrazine was added to the mixture. After adding Hydrazine, 2 or 3 equal increments of HAuCl₄ were added and stirred for approximately 5 minutes. A red solution similar to that of the gold control particles was observed. The mixture was poured into a glass bottle filled with argon gas, and later stored in a cold environment.

Synthesis of gold particles (used as control). A sodium citrate (Na₃C₆H₅O₇) procedure was used in the synthesis of gold nanobioparticles. To begin, 200 mL of distilled deionized water and 0.5 mL of 4% HAuCl₄ were mixed. The water/gold salt solution was then boiled using a condenser. While boiling, 3.8 mL of 1% sodium citrate was added. The amount of sodium citrate added controls the size of the particles; adding additional citrate results in a more rapid reduction and hence increases gold particle nuclei. This yields more but smaller particles. The amount used in the experiment gave approximately 20 nm gold particles. The solution was then boiled a second time for 20 minutes during which the gold solution turned a cherry red color. Finally, the solution was allowed to cool, poured into a glass bottle, and stored in a cold environment.

Conjugation of antibodies to core/shell particles (also used for Au particles). A sample of cAu was kept at pH 7.4 and a sample of cFe/cAu at approximately 7.5 (the cFe becomes unstable at lower pH so a higher pH is necessary for the cFe). In order to determine the concentration of immunoglobulin G (IgG) needed to coat the particles, increments of 5 μl IgG were each added to 200 μl aliquots of cAu/cFe. Twenty μl of dissolved NaCl was added to the cAu solutions, and 20 μl of CaCl₂ to the cFe, in order to indicate sufficient amounts of antibody. Mineral salts such as NaCl, CaCl₂ or AlCl salts neutralize the negative repulsive charges on the particle surfaces in proportion to the sixth power of the valence of the salt. Neutralization of the particle surface charge allows the particles to come close enough so that Van der Waals forces cause them to aggregate and the suspension turns from the reddish orange to blue. With an adequate amount of IgG, the particles are completely surrounded by antibodies. The antibodies keep the particles apart and replace the negative charge hence addition of salt does not result in particle aggregation and the color remains reddish orange. If no color change is seen after addition of salt sufficient antibody has been added to completely cover the particles. A concentration isotherm with increasing amounts of antibody is used to determine the point at which complete coverage occurs without excess antibody. For example, if, on addition of salt, a 10 μl IgG solution turned blue, while the 15 μl IgG solution remained red, 12 and 15 μl aliquots would be prepared. After making the additional aliquots, the lowest possible amount of antibody that resulted in the solution remaining red was then selected for large scale preparation. Using the acquired proportion from the above procedure, approximately 10 ml of each of the colloidal labels was prepared.

Confocal fluorescence microscopy techniques. In order to determine if the antibodies labeled the yeast cells primary anti-yeast antibody was labeled with a second antibody conjugated to Alexa Fluor 594. A rhodamine filter was used with the microscope in order to illuminate the marker with excitation wavelength and to view at the dye emission wavelength. Glass slides were prepared, covered, and sealed with nail polish to prevent disassembly due to the inverted objective lens on the microscope.

Scanning electron microscopy (SEM) techniques. An SEM was used to examine labeling of the yeast, i.e. attachment of the magnetite/gold core/shell particles, conjugated directly to the anti-yeast antibody, and the gold nanobioparticles also conjugated directly to the anti-yeast antibody. Cells with attached nanobioparticles were first washed with 0.1 M phosphate buffer solution (PBS) and fixed with 4% formaldehyde and 1% glutaraldehyde. The samples were then dehydrated with an increasing gradient of ethanol solutions (30%, 50%, 70%, 80%, 90%, 95%, 100%, dehydrated with 100% twice). The critical point procedure (Tousimis 780A Critical Point Dryer) was used to dry the samples; 100% ethanol was used as the intermediate fluid and liquid CO₂ was the transitional fluid. An IBS/TM 200S Ion Beam Sputter coater was then used to coat the samples with 3 nm of platinum. The platinum coating grounds samples eliminating charging. It also increases the ratio of secondary to primary electrons and hence improves the signal to noise ratio in the SEM. In addition, the platinum coating reduces the effects of heat on the sample cells.

Heating labeled cells using an oscillating magnetic field. An apparatus as explained above was used to provide a source for the oscillating magnetic field.

Controls used in experiments. Controls to determine if the magnetic field or if the antibody-particle conjugates affected yeast viability included tests of C. albicans cells exposed to the magnetic field with no attached particles as well as cells conjugated to non-magnetic gold nanobioparticles. Also, examination of the C. albicans unlabelled and labeled with cAu and with cFeAu but not exposed to an oscillating magnetic field was performed examined to measure any effects of the labeling alone.

The categories of cAu and cFeAu that were not exposed to the magnetic field “no magnetic field” were the controls for the magnetic field treated and the gold was the control for the effects of the magnetic field on the yeast since there is no or little effect on the gold. In initial studies looking at unlabeled yeast the usual number of dead cells was in the 3% to 5% range, therefore unlabeled yeast was not included in the controls. Killing of yeast cells at levels over the controls clearly occurs when inductive heating of the cFeAu is employed, however the killing of yeast cells is not 100%.

A control test for the Trypan blue indicator was administered to examine its ability to dye the dead yeast cells. A test tube of broth solution with suspended yeast cells was heated in boiling water for approximately 40 seconds. A slide was then prepared with a proportionate amount of trypan blue, and examined under a microscope.

Approximately 7×10⁶ actively growing Candida albicans yeast were labeled with rabbit anti-Candida antibody conjugated to either colloidal gold or colloidal magnetite-gold particles. Cells were left unexposed or exposed to an oscillating magnetic field of 0.008 T at 500 kHz for 10 minutes (5 treatments of 2 minutes each). This work was performed on an air-cooled solenoid magnetic field generator. Since some coil heating and associated loss of field strength occurred, the average T for the 2 minute period was approximately 0.007 as the T at the end of each 2 minute run would drop to a minimum of 0.0055T. This adversely affected the total heating. Cell viability was determined using trypan blue exclusion. Between 100 and 150 cells were counted for each treatment. The results are shown in Table 5.

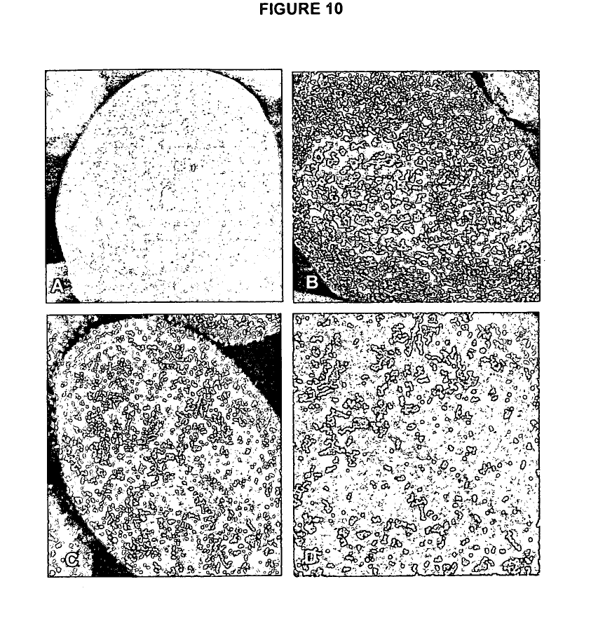

FIG. 10 shows SEM images of the surfaces of three yeast cells. Shown in FIG. 10A is the surface of an unlabeled control yeast cell at 20,000× magnification. Shown in FIG. 10B is the surface of a yeast cell labeled with nanobioparticles at 20,000× magnification. Shown in FIG. 10C is the surface of a yeast cell that has been labeled and exposed to the oscillating magnetic field, also at 20,000× magnification. In the first two yeasts (FIG. 10A, B) the cell membranes are normal and entire without any holes. In the third image (FIG. 10C) it is apparent there are a number of small holes in the membrane, dotting the surface. The fourth image (FIG. 10D) is an image of the same cell as in “C” except at higher magnification (40,000× magnification) to more clearly show the holes all over the surface. Inductive heating of core/shell particles substantially increased killing over controls. With yeast cells, the killing is significant but lower than the virtually complete (100%) killing that was seen with less durable mammalian cells that have been labeled and exposed to the oscillating magnetic field. It may be possible to modify the system with respect to particle size, labeling numbers, length of treatment, and field generator design. Such modifications may increase the percentage of killed yeast cells.

TABLE 5
Summary of experiments with yeast cells

| | |
|---|---|
| Experiment | Result |

Experiment 1 with 10 nm particles, >99% cells labeled

| | | |
|---|---|---|
| No labels - yeast heated 40 sec in boiling H₂O | 100% | dead |
| cAu - no magnetic field | 3.9% | dead |
| cAu - + magnetic field | 4.4% | dead |
| cFe—Au - no magnetic field | 3.1% | dead |
| cFe—Au -magnetic field | 12.8% | dead |

Experiment 2 with 20 nm particles, >99% of cells labeled

| | | |
|---|---|---|
| No labels - yeast heated 40 sec in boiling H₂O | 100% | dead |
| cAu - no magnetic field | 3.0% | dead |
| cAu - + magnetic field | 6.3% | dead |
| cFe—Au - no magnetic field | 11.6% | dead |
| cFe—Au - + magnetic field | 42.1% | dead |

Experiment 3 with 20 nm particles, >99% cells labeled

| | | |
|---|---|---|
| cAu - no magnetic field | 12.4% | dead |
| cAu - + magnetic field | 7.2% | dead |
| cFe—Au - no magnetic field | 9.6% | dead |
| cFe—Au - + magnetic field | 27.9% | dead |

Experiment 4 with 20 nm particles, >99% cells labeled

| | | |
|---|---|---|
| No labels - yeast heated 40 sec in boiling H₂O | 80% | dead |
| cFe—Au - + magnetic field | 38.8.0% | dead |

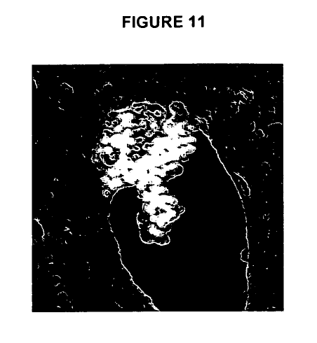

Cell-specific targeting results and inductive heating result in individual cell membrane rupture (“membrane melting”). FIG. 11 shows an SEM micrograph of a prostate cancer cell exposed to Oscillating Magnetic Field with a frequency of 500 kHz at 0.008 Tesla for 2.5 minutes. Prostate cancer cells were labeled by goat anti PAP IgG then further incubated with donkey anti goat secondary antibody conjugated to 15 nm core shell (cFeAu). The magnification is 200,000×. An apparent hole on the cell surface was created in the area where the beads are attached. The rest of the membrane structure is still intact. The sample was preserved well so that the dangling nanobioparticles still remain on the cell membrane. This example illustrates the mechanism of how, by heating the nanobioparticles, the cell membrane is locally punctured, which leads to cell death. Thus, using the compositions and methods of the present invention, there is no bulk “cooking” of multiple cells. Instead, the membrane of individual cells may be damaged through the observed “membrane melting” effect (FIG. 11). The brighter dots in FIG. 11 are individual nanobioparticles.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of bioengineering, molecular biology, immunology, and colloidal chemistry, are within the scope of this invention. All publications, patents, and patent applications cited herein are incorporated by reference in their entirety for all purposes.

What is claimed is:

1. A system for killing a cell, comprising:
a) a colloidal particle that has a size of between about 1 nm and 100 nm, the particle comprising
b) a magnetic material, a shell that surrounds the magnetic material, and a biomolecule attached to the shell; and
c) a magnetic field generator for inductively heating the colloidal particle,
wherein the colloidal particle is capable of associating with the cell and is capable of being inductively heated by the magnetic field generator to a temperature capable of killing the cell.

2. The system of claim 1, which does not kill a second cell that is in contact with the cell that is associated with the colloidal particle.

3. The system of claim 1 wherein the shell comprises a thermosensitive polymer.

4. The system of claim 1 wherein the biomolecule is cell-specific.

5. The system of claim 1 wherein the magnetic material is selected from the group consisting of iron, cobalt, zinc, or nickel.

6. The system of claim 1 wherein the shell is selected from the group consisting of gold, silver, palladium, platinum, rhodium, molybdenum, and ruthenium.

7. The system of claim 1 wherein the biomolecule is a protein, a peptide, an antibody, a nucleic acid, an antigen, a glycoconjugate, a cellular surface molecule, a cellular component, a toxin, or a drug.

8. The system of claim 1 wherein the magnetic material is iron or iron oxide, the shell is gold, and the biomolecule is an antibody specific for the cell.

9. The system of claim 1 wherein the biomolecule associates with a second biomolecule, the second biomolecule being a membrane molecule and the second molecule being specific for the cell.

10. A method for killing a cell, the method comprising:
a) providing a colloidal particle that has a size of between about 1 nm and 100 nm, the particle comprising:
i. a core that comprises a magnetic material,
ii. a shell that surrounds the magnetic material, and
iii. a biomolecule attached to the shell;
b) contacting the colloidal particle with the cell such that the colloidal particle associates with the cell; and
c) inductively heating the colloidal particle with a magnetic field to a temperature that will kill the cell.

11. The method of claim 10 wherein a second cell that is in contact with the cell that is contacted with the colloidal particle is not killed when the colloidal particle is inductively heated.

12. The method of claim 10 wherein the shell comprises a thermosensitive polymer.

13. The method of claim 10, wherein the biomolecule is cell-specific.

14. The method of claim 10, wherein the biomolecule associates with a second biomolecule, the second biomolecule being a membrane molecule and the second molecule being specific for the cell.

15. The method of claim 10, wherein the biomolecule is a protein, a peptide, an antibody, a nucleic acid, an antigen, a glycoconjugate, a cellular surface molecule, a cellular component, a toxin, or a drug.

16. A delivery system for delivering a biomolecule to a cell, the delivery system comprising:
a) a colloidal particle-biomolecule conjugate, wherein the colloidal particle has a size of between about 1 nm and 100 nm, the colloidal particle comprising:
i. a core that comprises a magnetic material, and
ii. a shell that surrounds the core,
wherein the biomolecule is a protein, a peptide, an antibody, a nucleic acid, an antigen, a glycoconjugate, a cellular surface molecule, a cellular component, a toxin, or a drug; and
b) administration means for administering the colloidal particle-biomolecule conjugate to the cell.

17. The delivery system of claim 16, wherein the core comprises a core magnetic material selected from the group consisting of iron, cobalt, zinc, or nickel.

18. The delivery system of claim 16, wherein the shell comprises a metal selected from the group consisting of gold, silver, palladium, platinum, rhodium, molybdenum and ruthenium.

19. The delivery system of claim 16, wherein the shell comprises a thermosensitive polymer.

20. The delivery system of claim 16, which does not deliver a biomolecule to a second cell that is in contact with the cell that is associated with the colloidal particle, when the colloidal particle-biomolecule conjugate is administered to the cell.

21. The delivery system of claim 16, wherein the administration means comprises inductively heating the magnetic material using a magnetic field.

22. A method for delivery of a biomolecule to a specific cell, the method comprising:
a) providing a colloidal particle that has a size of between about 1 nm and 100 nm and that comprises
i. a magnetic material and
ii. a substance that comprises a biomolecule, the substance surrounding the magnetic material;
b) contacting the colloidal particle with the cell such that the colloidal particle associates with the cell; and
c) inductively heating the colloidal particle using a magnetic field to a temperature which delivers the biomolecule to the cell.

23. The method of claim 22 wherein the biomolecule is a protein, a peptide, an antibody, a nucleic acid, an antigen, a glycoconjugate, a cellular surface molecule, a cellular component, a toxin, or a drug.

24. The method of claim 22 wherein the biomolecule is not delivered to a second cell that is in contact with the cell that is contacted with the colloidal particle, when the colloidal particle is inductively heated.

25. The method of claim 22 wherein the substance that surrounds the magnetic material comprises a thermosensitive polymer.
